SEQUENCE LISTING (20 sequences)

SEQ ID NO: 1 - DNA, 19 bp, synthetic construct
  source 1..19: mol_type = other DNA
cccactcctccacctttga

SEQ ID NO: 2 - DNA, 19 bp, synthetic construct
  source 1..19: mol_type = other DNA
ccaccctgttgctgtagcc

SEQ ID NO: 3 - DNA, 18 bp, synthetic construct
  source 1..18: mol_type = other DNA
agctgaacaggaacaacg

SEQ ID NO: 4 - DNA, 19 bp, synthetic construct
  source 1..19: mol_type = other DNA
cttcatggtgcccgtggtc

SEQ ID NO: 5 - DNA, 22 bp, synthetic construct
  source 1..22: mol_type = other DNA
tttacaacaaatacccagatgc

SEQ ID NO: 6 - DNA, 21 bp, synthetic construct
  source 1..21: mol_type = other DNA
atggctttcgttggacttact

SEQ ID NO: 7 - DNA, 20 bp, synthetic construct
  source 1..20: mol_type = other DNA
gactgtgacgagttggctga

SEQ ID NO: 8 - DNA, 20 bp, synthetic construct
  source 1..20: mol_type = other DNA
ctggagaggagcagaactgg

SEQ ID NO: 9 - DNA, 21 bp, synthetic construct
  source 1..21: mol_type = other DNA
tatccccaacatgcccattcg

SEQ ID NO: 10 - DNA, 22 bp, synthetic construct
  source 1..22: mol_type = other DNA
tggtaggcaaagtagtacagcc

SEQ ID NO: 11 - DNA, 22 bp, synthetic construct
  source 1..22: mol_type = other DNA
atgacaccaaaaccctcatcaa

SEQ ID NO: 12 - DNA, 21 bp, synthetic construct
  source 1..21: mol_type = other DNA
gaagtccaaaccggtgacttt

SEQ ID NO: 13 - DNA, 21 bp, synthetic construct
  source 1..21: mol_type = other DNA
cctgaaacttcaagagtacca

SEQ ID NO: 14 - DNA, 21 bp, synthetic construct
  source 1..21: mol_type = other DNA
tcatgaggcttattgtagagc

SEQ ID NO: 15 - DNA, 21 bp, synthetic construct
  source 1..21: mol_type = other DNA
ctgatacccttgagacacctg

SEQ ID NO: 16 - DNA, 22 bp, synthetic construct
  source 1..22: mol_type = other DNA
gggctctgatctctgcatctac

SEQ ID NO: 17 - DNA, 22 bp, synthetic construct
  source 1..22: mol_type = other DNA
gggctctgatctctgcatctac

SEQ ID NO: 18 - DNA, 21 bp, synthetic construct
  source 1..21: mol_type = other DNA
gcagtcgcagttttcacactc

SEQ ID NO: 19 - DNA, 20 bp, synthetic construct
  source 1..20: mol_type = other DNA
tggtgccgagtgagccgaag

SEQ ID NO: 20 - DNA, 23 bp, synthetic construct
  source 1..23: mol_type = other DNA
agtggttggttaataagccgaag

(19) United States
(12) Patent Application Publication
(10) Pub. No.: US 2024/0368538 A1
(43) Pub. Date: Nov. 7, 2024

(54) COMPOSITIONS AND METHODS FOR PROTECTING ANIMAL CELLS FROM COMPRESSIVE FORCES

(71) Applicant: Trustees of Tufts College, Medford, MA (US)

(72) Inventors: Onur Hasturk, Medford, MA (US); David L. Kaplan, Concord, MA (US)

(21) Appl. No.: 18/666,235

(22) Filed: May 16, 2024

Related U.S. Application Data
(63) Continuation of application No. PCT/US2022/081216, filed on Dec. 8, 2022.
(60) Provisional application No. 63/265,155, filed on Dec. 8, 2021.

(51) Int. Cl. C12N 5/00; C12N 5/077; C12N 5/0775; C12N 5/079

(52) U.S. Cl. CPC C12N 5/0012; C12N 5/0018; C12N 5/0618; C12N 5/0656; C12N 5/0663; C12N 2500/50; C12N 2501/25; C12N 2525/00; C12N 2533/50; C12N 2533/74; C12N 2537/10

(57) ABSTRACT

A composition is disclosed including a composite microbead that has one or more animal cells embedded therein. The composite microbead has a material matrix composed of silk fibroin and a mixture of modified and unmodified alginate. The composite microbead has reduced disintegration by ion exchange when compared with material lacking the modified alginate. The material matrix is ionically and covalently crosslinked. The animal cells can be on-demand released by contact with a calcium chelator and a reducing agent. The microbeads can be made by introducing droplets of a pre-hydrogel solution including the cells into a crosslinking solution. The resulting microbeads provide the animal cells with an enhanced ability to survive elevated pressures, immunocamouflaging, permselectivity against higher molecular weight molecules, protection from harsh chemical environments, and protection from UV radiation.

### CROSS-REFERENCE TO RELATED APPLICATIONS

This application is a continuation of International Application Serial Number PCT/US2022/081216, filed Dec. 18, 2022. International Application Serial Number PCT/US2022/081216 is related to and claims priority to U.S. Provisional Patent Application No. 63/265,155, filed Dec. 8, 2021. Each of the foregoing patent applications is incorporated herein by reference in their entirety for all purposes.

### STATEMENT REGARDING FEDERALLY FUNDED RESEARCH

This invention was made with government support under grant EB027062 awarded by the National Institutes of Health and grant 2104294 awarded by the National Science Foundation. The government has certain rights in the invention.

### SEQUENCE LISTING

A Sequence Listing accompanies this application and is submitted as an XML file of the sequence listing named “T002566 US-2095.0610 Sequence Listing” which is 19 kbytes in size and was created on May 13, 2024. The sequence listing is electronically submitted with the application and is incorporated herein by reference in its entirety.

### BACKGROUND

The last two decades of regenerative medicine and biotechnology have made extensive use of mammalian cells for production of biopharmaceuticals and transplantation therapies. Proper folding and posttranslational modifications of recombinant proteins necessitate the use of eukaryotic cells, while self-renewing capacity and multilineage differentiation of stem cells attract attention in tissue engineering-inspired or cell transplantation therapies. Mesenchymal stem cells (MSCs) hold promise in ex vivo engineering of tissues and organs as well as in cell therapy for immunomodulation to reduce inflammation and promote tissue repair in rheumatoid arthritis, spinal cord injuries, myocardial infarctions, Crohn's Disease, or cartilage and meniscus repairs, among other needs. Similarly, neural progenitor cells (NPCs) have been utilized for neural tissue engineering and cell transplantation therapies for diseases affecting the central and peripheral nervous systems including spinal cord injuries, brain tumors and Parkinson's disease. Use of mammalian cells involves large numbers of cells cultured and processed ex vivo and then delivered in vivo, subjecting cells to varying degrees of environmental stress, including polycations, UV irradiation, attachment deprivation (anoikis), non-physiological pH, or inflammatory cytokines.

Mammalian cells lacking a protective cell wall or exoskeleton being surrounded only by a fragile lipid membrane, however, renders them particularly more susceptible to mechanical stress applied during in vitro processing in bioreactors, or during centrifugation, delivery through capillaries, and blood circulation after in vivo transplantation. In the regenerative medicine applications, two specific examples of where cells could be exposed to extremes of mechanical stress are injection-based cell delivery and 3D bioprinting. Extrusion in a viscous carrier gel or bioink solution through a fine (small gauge) needle that is favored to minimize tissue damage and patient discomfort during injection-based cell transplantation, or to maximize resolution during 3D bioprinting could subject cells to extremes of mechanical stress that can reach up to hundreds of kPa, which impact cell fate and can cause necrotic death by disrupting the cell membrane. Impaired cell viability due to stretching by extensional forces originating from abrupt increases in linear velocity when the fluid enters a smaller cross-sectional area combined with membrane rupture by high levels of shear forces at the needle walls reduces production yield or the rate of therapeutic success, and causes acute inflammatory responses at the implantation site. Thus, it is essential to provide protection against environmental stresses to preserve cell viability and function during ex vivo processing and delivery, prompting opportunities to toughen or to bring a bionic status to mammalian cells.

A need exists for technologies that can strengthen the ability of animal and mammalian cells to withstand hydrodynamic forces.

### SUMMARY

In one aspect, the present disclosure provides a composition comprising a composite microbead having embedded therein one or more animal cells. The composite microbead has a material matrix that is composed of silk fibroin and a mixture of modified and unmodified alginate. The composite microbead has reduced disintegration by ion exchange when compared with a comparison microbead. The comparison microbead replaces the modified alginate with an equal amount by weight of the unmodified alginate, such that the comparison microbead includes no unmodified alginate. The silk fibroin, the modified alginate, and the unmofieid alginate are covalently and ionically crosslinked. The one or more animal cells can be on-demand released from the composite microbead by contacting the composite microbead with a mixture of a calcium chelator and a reducing agent.

In another aspect, the present disclosure provides a method of microencapsulating animal cells. The method includes: a) suspending a plurality of the animal cells in a pre-hydrogel solution comprising silk fibroin, modified alginate, and unmodified alginate and optionally further comprising a tyramine-modified gelatin; b) introducing microdroplets of the pre-hydrogel solution into a crosslinking solution, thereby microencapsulating at least a portion of the plurality of the animal cells into a composite microbead. The pre-hydrogel solution and/or the crosslinking solution includes horseradish peroxidase. The crosslinking solution comprises an ionic crosslinker.

### DETAILED DESCRIPTION

Before the present invention is described in further detail, it is to be understood that the invention is not limited to the particular embodiments described. It is also understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

Specific structures, devices and methods relating to modifying biological molecules are disclosed. It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. When two or more ranges for a particular value are recited, this disclosure contemplates all combinations of the upper and lower bounds of those ranges that are not explicitly recited. For example, recitation of a value of between 1 and 10 or between 2 and 9 also contemplates a value of between 1 and 9 or between 2 and 10.

As used herein, “silk fibroin” refers to silk fibroin protein whether produced by silkworm, spider, or other insect, or otherwise generated (Lucas et al., Adv. Protein Chem., 13:107-242 (1958)). Any type of silk fibroin can be used in different embodiments described herein. Silk fibroin produced by silkworms, such as Bombyx mori, is the most common and represents an earth-friendly, renewable resource. For instance, silk fibroin used in a silk film may be attained by extracting sericin from the cocoons of B. mori. Organic silkworm cocoons are also commercially available. There are many different silks, however, including spider silk (e.g., obtained from Nephila clavipes), transgenic silks, genetically engineered silks, such as silks from bacteria, yeast, mammalian cells, transgenic animals, or transgenic plants, and variants thereof, that can be used. Sec, e.g., WO 97/08315 and U.S. Pat. No. 5,245,012, each of which is incorporated herein by reference in their entireties.

The present disclosure provides a composition. The composition includes a composite microbead having embedded therein one or more animal cells. The composite microbead includes a material matrix. The material matrix is composed of silk fibroin and a mixture of modified and unmodified alginate. The composite microbead has reduced disintegration by ion exchange when compared with a comparison microbead. The comparison microbead replaces the modified alginate with an equal amount by weight of the unmodified alginate, such that the comparison microbead include no unmodified alginate. The silk fibroin, modified alginate, and unmodified alginate are covalently and ionically crosslinked. The one or more animal cells can be on-demand released from the composite microbead by contacting the composite microbead with a mixture of a calcium chelator and a reducing agent.

The composite microbead can have a diameter of between 1 μm and 1 mm or between 100 μm and 500 μm. In some cases, the composite microbead can have a diameter of at least 1 μm, at least 10 μm, at least 50 μm, at least 100 μm, or at least 250 μm. In some cases, the composite microbead can have a diameter of at most 1 mm, at most 750 μm, or at most 500 μm.

The composite microbead has an elastic modulus that is greater than an elastic modulus of the comparison microbead. Without wishing to be bound by any particular theory, it is believed that this enhanced elastic modulus contributes significantly to the ability of the present composition to protect cells that would otherwise be fatally damaged by a specific mechanical force.

The composite microbead provides permselectivity against large macromolecules, while allows for free diffusion of nutrients and gases. The mesh size of the microbeads defined by the crosslinking density of the hydrogel matrix limits the diffusion of macromolecules with molar mass above a threshold value, around 10 kDa in this specific case, into the core of the microbead. Permselectivity could be tuned by modulating the extent of ionic and covalent crosslinking of the hydrogel microbeads. Limited diffusivity of large molecules such as polymer chains and cytokines into the microbeads provides protection to encapsulated cells against potential cytotoxic effects of these molecules.

The modified alginate can be modified to include phenol moieties to enable enzymatic (covalent) crosslinking with other modified alginate chains or silk fibroin. In some cases, the modified alginate is an alginate conjugated with disulfide-linked phenol moieties.

The silk fibroin can in some cases be enhanced to possess more phenol moiety than native silk fibroin. In these cases, the silk fibroin is a modified silk fibroin including tyramine substitution in addition to intrinsic tyrosine moieties.

The material matrix can contain the unmodified alginate in an amount by weight excluding water of the material matrix of between 10% and 40%, the modified alginate in an amount by weight excluding water of the material matrix of between 10% and 50%, and the silk fibroin in an amount by weight excluding water of the material matrix of between 30% and 75%.

In some cases, the material matrix can further include a gelatin component to further enhance the bioactivity, elastic modulus, and other protective properties. In these cases, the gelatin is modified for covalent crosslinking. The material matrix can further comprise a tyramine-modified gelatin. In these cases, the silk fibroin, the modified alginate, the unmodified alginate, and the tyramine-modified gelatin are covalently crosslinked. In cases where the tyramine-modified gelatin is present, the material matrix can contain the alginate in an amount by weight excluding water of the material matrix of between 10% and 40%, the modified alginate in an amount by weight excluding water of the material matrix of between 10% and 50%, the silk fibroin in an amount by weight excluding water of the material matrix of between 30% and 75%, and the tyramine-modified gelatin in an amount by weight excluding water of the material matrix of between 1% and 40%.

The primary distinguishing feature of the animal cells is the lack of a cell wall, which is present in many unicellular eukaryotes, fungi, and plant cells, and can provide significant structural strength to the cell as a result. In some cases, the animal cells are mammalian cells. In some cases, the animal cells are murine cells, such as L929 murine fibroblasts. In other cases, the animal cells can be human cells, such as bone marrow-derived mesenchymal stem cells and neural progenitor cells. The animal cells can be any human adherent cells such as fibroblasts and tissue specific primary cells including osteoblasts, adipocytes, and myocytes, or blood cells such as monocytes, T cells, B cells, or red blood cells.

In addition to providing enhanced structural stability to the animal cells, the material matrix also immunocamouflages the cells, thereby at least partly preventing binding to surface markers of the cells. This provides the ability to deliver cells to an environment where immunological systems might otherwise prevent their passage.

The present disclosure also provides a method of microencapsulating animal cells. The method includes: a) suspending a plurality of the animal cells in a pre-hydrogel solution comprising silk fibroin, modified alginate, and unmodified alginate; and b) introducing microdroplets of the pre-hydrogel solution into a crosslinking solution, thereby microencapsulating at least a portion of the plurality of the animal cells into a composite microbead. The pre-hydrogel solution and/or the crosslinking solution includes horseradish peroxidase (HRP). The crosslinking solution comprises an ionic crosslinker and hydrogen peroxide (H₂O₂). The aspects of the present disclosure described above with respect to the composition are includable in the methods described herein and vice versa. Similarly, the aspects describe above with respect to the composition are combinable with features described in the method. Unless the context clearly dictates otherwise, the composition and method are two descriptions of fundamentally the same thing.

The methods can further include subjecting the composite microbead to elevated pressure. The elevated pressure is fatal to the animal cells in isolation from the composite microbead, but the animal cells embedded in the composite microbead can survive the elevated pressure. Examples of situations where the cells may experience elevated pressure include, but are not limited to, processing a recombinant protein, transplanting the composite microbead, tissue engineering with the composite microbead, injection-based delivery of the composite microbead, bioprinting the composite microbead.

The methods can further include culturing the embedded cells. This can be achieved generally by culturing the composite microbeads as though they were the cells themselves. The composition disclosed herein can protect the cells from culture conditions that otherwise would be fatal to the cells in isolation from the composite microbead. Examples of situations where the cells may experience otherwise fatal culturing conditions include, but are not limited to, culturing in the presence of polycationic polyethyleneimine, in the presence of apoptotic inflammatory cytokine tumor necrosis factor (TNF)-α, and/or at extracellular acidosis at a pH of 5.0.

The methods can further include exposing the composite microbead to UV-C light at 254 nm. This radiation would typically be fatal to the animal cells at sufficient radiation levels, but the composite microbead can provide protection from these rays.

The methods can further include initiating dissolution of the composite microbead and releasing the at least a portion of the plurality of the animal cells. Initiating dissolution can be achieved by contacting the composite microbead with a calcium chelator and a reducing agent. The calcium chelator can be sodium citrate, ethylenedioxy-diethleyene-dinitrilio-tetraacetic acid (EDTA), ethylene-glycol-bis-(2-aminocthyl)-N,N,N′,N′-tetraacetic acid (EGTA), or the like. In some specific embodiments, the calcium chelator is sodium citrate. The reducing agent can be glutathione.

Encapsulation of mammalian cells with hydrogel microbeads could provide protection against processing stress applied during biotechnology and regenerative medicine applications, including recombinant protein manufacturing, cell transplantation, and tissue engineering. The hydrogel formulations used for cell microencapsulation are largely dominated by ionically crosslinked alginate, which suffers from low structural stability under physiological culture conditions and poor cell-matrix interactions. In this disclosure, formulations for silk fibroin-based composite hydrogel microbeads are described for microencapsulation of mammalian cells for culture and cytoprotection. Alginate-templated, dual (ionically and enzymatically) crosslinked silk and silk/gelatin composite microbeads are fabricated via centrifugation-based droplet formation using cost-effective, commercially available consumable such as 34-gauge needles, poly(dimethylsiloxane) (PDMS) plugs, and 2 mL centrifugation tubes. Composite hydrogel microspheres with permanent covalent crosslinks using unmodified alginate, with on-demand cleavable crosslinks using alginate with phenol moieties conjugated using reduction sensitive linkers, synthesis of which is described in this disclosure for the first time. Enzymatic crosslinking is achieved by inclusion of horseradish peroxidase (HRP) in the pre-hydrogel solution and hydrogen peroxide in the crosslinking bath together with calcium chloride for ionic crosslinking of the alginate component. Composite hydrogel microbeads have an average diameter of 150-200 μm, and do not disintegrate after treatment with sodium citrate, a calcium chelator, unlike control microbeads fabricated using unmodified alginate only. Composite microbeads provide permselectivity against diffusion of high molecular weight molecules and display significantly higher elastic modulus than an alginate-only control. L929 murine fibroblasts and bone marrow-derived human mesenchymal stem or neural progenitor cells were successfully encapsulated in composite microbeads, and retained high viability after on-demand release by treatment with a mixture of sodium citrate and glutathione (a mild reducing agent). Composite microbeads provide protection to encapsulated cells against mechanical stress during extrusion through needles with small inner diameter, and preserve cell growth and differentiation, suggesting potential applications in injection-based delivery and 3D bioprinting of mammalian cells with higher success rates. Moreover, encapsulated cells survive harsh culture conditions, including incubation with high molecular weight polycation polyethylenciminc (PEI), apoptotic inflammatory cytokine tumor necrosis factor (TNF)-α, culture at extracellular acidosis at pH 5.0, attachment deprivation (anoikis) in suspension, and exposure to UV-C radiation at 254 nm.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1

L929 fibroblasts from mouse subcutaneous connective tissues or bone marrow-derived human MSCs and NPCs (ATCC, Manassas, VA) were microencapsulated in dual crosslinked, on-demand degradable composite hydrogel microbeads using centrifugation-based droplet fabrication. Briefly, cells were suspended at a concentration of 10⁷ cells/mL in pre-hydrogel solutions composed of 1.5% w/v alginate and 2% w/v Alg-DSP with 3.5% w/v SF-TA, or with 2.5% w/v SF-TA and 1% G-TA w/v supplemented with 100 U/mL HRP in HD buffer (40 mM HEPES with 5% w/v dextrose, pH 7.4). 10 μL of cell-laden solution was centrifuged through a 34 G needle at 2,250 g for 1 min into crosslinking solution (40 mM HEPES, 4.5 g/mL dextrose, 150 mM NaCl, 5 mM KCl, 50 mM CaCl₂) and 0.5 mM H₂O₂). The crosslinking solution was discarded immediately after centrifugation and the microbeads were resuspended in culture media.

Uncoated and microencapsulated cells were incubated in 0.1 mg/mL PEI solution in 1×PBS for 10 min or in pH 5.0 MES buffered saline solution (10 mM MES, 137 mM NaCl, 3 mM KCl) for 30 min to evaluate cytoprotection against polycations and acidic environment, respectively. To investigate the protection against UV irradiation, microbeads suspended in 1×PBS were exposed to UV-C light at 254 nm using an 8 Watt UVP UVLMS-38 UV lamp (Analytik Jena AG, Thuringia, Germany) for 10-, 20-, or 30-min. Cells were cultured on PDMS-coated well plates or in FBS-free growth media supplemented with 40 ng/ml of TNF-α for 48 hours at 37° C. with 5% CO₂ to assess protection against attachment deprivation or apoptotic factors, respectively. Immediately after exposure to environmental stress, control cells and the cells released from the hydrogel microbeads by sodium citrate-GSH treatment were seeded in well plates at a density of ˜3×10⁵ cells/cm² for the analysis of survival rates.

For antibody labeling, 2×105 hMSCs resuspended in flow cytometry staining (FCS) buffer (1×PBS with 1:1000 sodium azide and 1:100 BSA) were incubated with mouse IgG1 anti-CD44 (ab9524, 0.1 μg) or anti-CD90 (ab23894, 1 μg) for 1 h at RT. After 3× washing, cells were incubated with FITC-coupled goat anti-mouse IgG1 (ab97239, 1:100) secondary antibody for 1 h at RT. Cells incubated with secondary antibody only or with mouse IgG1 K isotype control (ab170190, 0.2 μg) followed by secondary antibody staining were used as negative controls. Flow cytometry analysis was performed using a FACSCalibur modular flow cytometer (BD Biosciences, Franklin Lakes NJ). After antibody labeling, control and microencapsulated cells were fixed in 4% paraformaldehyde and imaged under confocal microscope.

Cells were suspended in 6% w/v PEG solution in culture media at a density of 5×10⁶ cells/mL and 420 μL of cell suspension (n=3) was loaded onto the Peltier of ARES-LS2 rheometer. The 25 mm cone was lowered to 47 μm and the cell suspension was exposed to a constant shear rate of 2000 s⁻¹ for 5, 60, 150 or 300 s at 24° C.

Cells were suspended in 6% (w/v) PEG solution at a density of 5×10⁶ cells/mL and 400 uL of cell suspension was loaded into 1 mL syringes (BD Biosciences, San Jose, CA). Cells were extruded through 1.27 cm long 27-gauge (BD, Franklin Lakes, NJ) needles (inner diameter of 210 μm) at a flow rate of 3 mL·min⁻¹ using a Harvard PHD 2000 syringe pump (Harvard Apparatus, Holliston, MA). Assuming that the PEG solution was incompressible with no volume change and laminar flow inside the nozzle with no change in velocity profile with time, the wall shear stress was estimated using equation (1):

$$τ = {m [ \frac{Q}{π ( \frac{n}{3 n + 1} ) ( {R}^{\frac{3 n + 1}{n}} )} ]}^{n} \cdot R \qquad ( 1 )$$

where Q is the flow rate and R is the inner radii of the nozzle. The minimum residence time in the needle (t) was estimated using equation (2):

$$t = \frac{L π {R}^{2}}{Q} \qquad ( 2 )$$

where L is the needle length.

50 μL of cell suspension after syringe-based extrusion or constant shearing using a rheometer was transferred into 200 μL of culture media without FBS. 10⁴ cells in 10 μL suspension were then seeded into 96 well (3.125×10⁴ cells/cm²) for LDH, live/dead and apoptosis assays or into 48 well plates (10⁴ cells/cm²) for metabolic activity assay. The activity of extracellular LDH released from damaged cells was quantified using a LDH cytotoxicity detection kit (Roche, Indianapolis, IN) according to the manufacturer's instructions. Cell viability was assessed using Live/Dead assay kit (Invitrogen, Carlsbad, CA) 2 h after seeding and imaged using a BZ-X700 Fluorescence Microscope (Keyence Corp., Itasca, IL). Five random images were taken (n=3) and analyzed using live/dead staining macro of ImageJ to estimate the % viability of the released cells. Metabolic activity of the cells was determined by % dye reduction (n=4) using alamarBlue metabolic activity assay (Invitrogen, Carlsbad, CA).

Total RNA extraction was performed using RNeasy Plus Micro Kit (Qiagen, Germantown, MD) according to manufacturer's instructions. First strand cDNAs were reverse transcribed using RevertAid First Strand cDNA Synthesis Kit (Thermo Fisher Scientific, Rockford, IL) from each sample according to manufacturer's instructions using a thermal cycler (iCycler, BIO-RAD, USA). qPCR was performed using GoTaq® qPCR Mastermix (Promega, USA) according to manufacturer's instructions using forward and reverse primers (Table 1) specific for glyceraldehyde 3-phosphate dehydrogenase (GAPDH) as the housekeeping gene; alkaline phosphatase (ALP), osteopontin (OP) and osteocalcin (OC) as osteogenic markers; adiponectin, leptin and peroxisome proliferator activated receptor gamma (PPARg) as adipogenic markers; and neuroepithelial stem cell protein (Nestin), microtubule associated protein 2 (MAP2) and tubulin beta 3 class III (TUBB3) as neural markers. Relative expression of the genes compared to expansion media control was calculated using the ΔΔCt method (n=3) as described elsewhere.

TABLE 1

| Marker | | Sequence (5′-3′) |
|---|---|---|
| GAPDH | Forward | CCCACTCCTCCACCTTTGA |
| | Reverse | CCACCCTGTTGCTGTAGCC |
| ALP | Forward | AGCTGAACAGGAACAACG |
| | Reverse | CTTCATGGTGCCCGTGGTC |
| OP | Forward | TTTACAACAAATACCCAGATGC |
| | Reverse | ATGGCTTTCGTTGGACTTACT |
| OC | Forward | GACTGTGACGAGTTGGCTGA |
| | Reverse | CTGGAGAGGAGCAGAACTGG |
| Adiponectin | Forward | TATCCCCAACATGCCCATTCG |
| | Reverse | TGGTAGGCAAAGTAGTACAGCC |
| Leptin | Forward | ATGACACCAAAACCCTCATCAA |
| | Reverse | GAAGTCCAAACCGGTGACTTT |
| PPARg | Forward | CCTGAAACTTCAAGAGTACCA |
| | Reverse | TCATGAGGCTTATTGTAGAGC |
| Nestin | Forward | CTGCTACCCTTGAGACACCTG |
| | Reverse | GGGCTCTGATCTCTGCATCTAC |
| TUBB3 | Forward | GGCCAAGGGTCACTACACG |
| | Reverse | GCAGTCGCAGTTTTCACACTC |
| MAP2 | Forward | TGGTGCCGAGTGAGAAGAAG |
| | Reverse | AGTGGTTGGTTAATAAGCCGAAG |

Cells were seeded at a density of ˜4×10⁴ cells/cm² and after 48 h of culture, expansion media was replaced with neural differentiation medium (Neurobasal medium supplemented with 2% v/v B-27, 1× GlutaMAX and 1% P/S) for hNPCs and either with Gibco StemPro osteogenesis or adipogesis differentiation media (Thermo Fisher Scientific, Rockford, IL) for hMSCs. Induction medium was replaced every 3 days with fresh medium. After culture in neural an adipogenic differentiation media for 14 days and in osteogenic differentiation medium for 21 days, samples were fixed in 4% paraformaldehyde solution for 15 min and rinsed with 0.01 M PBS. hMSCs were stained with Alizarin Red S for calcium deposition or Oil Red O for formation of lipid droplets and imaged under a brightfield microscope. For the immunostaining of hNPCs, fixed samples were permeabilized in 1×PBS with 0.1% v/v Triton X-100 for 30 min and incubated in blocking solution (1% w/v BSA and 0.1% v/v Tween 20 in 1×PBS) at 37° C. for 1 h. Samples were incubated in primary antibody solution (Mouse anti-β-tubulin III, ab78078 in 0.1% w/v BSA solution) overnight at 4° C. and in secondary antibody (Goat anti-mouse IgG Alexa Fluor 64, Thermo Fisher Scientific, Rockford, IL) for 1 h at RT. Cell nuclei were stained with 4′,6-diamidino-2-phenylindole (DAPI) for 5 min at RT and the specimens were imaged under confocal microscope.

All data are expressed as mean±standard deviation for n≥3. GraphPad Prism (GraphPad Software, La Jolla, CA) was used to perform One- or Two-way analysis of variance (ANOVA) with Tukey's post hoc multiple comparison test to determine statistical significance (*p≤0.05, ** p≤0.01, *** p≤0.001).

To mimic the hydrodynamic forces applied during injection-based delivery or 3D bioprinting, cells suspended in 6% w/v PEG solution were extruded through 27 G needles at a flow rate of 3 mL·min⁻¹. The maximum shear stress value at the needle walls were estimated to be 24.2 kPa.

Live/dead fluorescent micrographs of the L929 fibroblasts revealed a dramatic increase in the number of dead cells after needle extrusion in PEG solution. The activity of LDH released from damaged cells into the extrusion media was analyzed to assess the loss of membrane integrity in response to mechanical stress during extrusion. The LDH activity increased 4.7 folds after extrusion compared to no ejection control, indicating significant membrane damage and leakage of cytosolic components. Only ˜2-10% of the cells survived the extrusion through 27 G needle at 3 mL/min.

hNPCs were also significantly affected by extrusion. Number of viable hNPCs decreased significantly after needle extrusion.

A similar trend was also recorded for hMSCs, viability of which was significantly compromised after needle extrusion.

Cells encapsulated in Alg/Alg-DSP/SF-TA composite microbeads were exposed to hydrodynamic forces by extrusion in 6% w/v PEG solution through 27 G needle at 3 mL/min, corresponding to an estimated maximum wall shear stress of 24.2 kPa. Live/dead micrographs taken after on-demand release of the cells showed significantly lower number of dead cells in the microencapsulated group compared to the controls. Quantification of survival rate revealed viabilities of ˜82%, 87% and 80% after extrusion for microencapsulated hNPCs, L929 fibroblasts and hMSCs, respectively, which were not significantly different than untreated controls.

Live/dead imaging of hNPCs after extrusion showed that cells in all groups were able to spread and proliferate over 3 weeks of culture, reaching confluency by day 21. Metabolic activity assay revealed that cell proliferation reached a plateau by day 14 and did not increase significantly from day 14 to 21. Metabolic activity of extruded cells on day 1, however, was significantly lower than untreated control. Despite having no significant difference on day 1, microencapsulated cells displayed higher metabolic activity than extrusion control group on day 7, suggesting faster proliferation of encapsulated cells during the first week of culture.

Unlike hNPCs, hMSCs in extrusion control group were not able to reach confluency over 21 days, while untreated control and microencapsulated extrusion groups did as observed on live/dead micrographs. This observation was supported by metabolic activity assay, where all groups but extrusion control were able to reach a plateau by day 14 with no statistical difference among them. Metabolic activity of the extrusion control group on day 1 was below the detection limit, while those of microencapsulated extrusion groups were significantly lower than that of untreated control.

The differentiation of hNPCs and hMSCs after extrusion was evaluated by gene expression analysis and histology or immunostaining. ˜5-, 9- and 49-fold increases were recorded in the expressions of neural marker genes nestin, TUBB3 and MAP2 by the untreated control group 14 days of culture in neurogenic induction medium. While there was no significant difference in the expression of Nestin and MAP2, cells in the extrusion control group had significantly lower expression of TUBB3, which was also visualized by immunostaining of the cells with anti-TUBB3 antibodies. Expression of all neural genes tested by the microencapsulated extrusion groups was comparable with the untreated control with no statistical difference, and the cells were positive for TUBB3 staining on day 14.

hMSCs used in this study were able to undergo osteogenic or adipogenic differentiation upon incubation in respective induction media. Untreated control cells displayed significant upregulation of ALP and OP genes by ˜12 and 7-folds, respectively, while the relative expression of OC increased only 1.4-fold compared to the expansion media control. Upon adipogenic induction, expression of adiponectin, leptin and PPARg by the untreated control cells increased drastically by ˜72000-, 231- and 32-folds, respectively, indicating commitment of the cells into adipose lineage. Differentiation into osteogenic and adipogenic lineages was also confirmed by histology micrographs showing deposition of calcium and formation of lipid droplets positive for alizarin red and oil red staining, respectively, which were missing in expansion media controls. Interestingly, osteogenic or adipogenic genes were not significantly upregulated for the cells in extrusion control group, and the cells were negative for calcium deposition or lipid droplets. Although ˜260-fold increase was recorded in the expression of adiponectin, it was more than 250-fold lower compared to the untreated control. Microencapsulated cells, on the other hand, displayed significant increases in the expression of bone and fat cell markers and were positive for alizarin or oil red staining upon osteogenic or adipogenic induction after extrusion, respectively.

Cytoprotection by microencapsulation in composite microbeads was evaluated through exposure of cell-laden beads to cytotoxic or apoptotic factors, followed by the analysis of cell viability for L929 fibroblasts, hNPCs, and hMSCs. None of the cell types survived incubation in 0.1 mg/mL 70 kDa PEI solution in PBS for 10 min, revealing its high cytotoxicity on mammalian cell suspensions. After encapsulation in Alg/Alg-DSP/SF-TA microbeads, on the other hand, 80%, 62%, and 74% of L929 fibroblasts, hNPCs and hMSCs survived incubation in PEI solution. Response to incubation with TNF-α for 48 h varied with the different cell types: only ˜6% of L929 fibroblasts and ˜18% of hNPCs survived the treatment, while ˜74%±4% of the hMSCs were viable after incubation. The viabilities of L929 fibroblasts, hNPCs and hMSCs microencapsulated in Alg/Alg-DSP/SF-TA/G-TA composite microbeads were ˜81%, 72% and 79%, respectively, clearly indicating the physical barrier effect of hydrogel microbeads against the apoptotic factor. None of L929 fibroblasts survived attachment deprivation, while ˜33% and ˜40% of hNPCs and hMSCs, respectively, remained viable by forming cell clusters. Viabilities of microencapsulated hNPCs, L929 cells and hMSCs after culture on PDMS were ˜80%, 82% and 79%, respectively, indicating structural support and survival signals provided by the composite microbeads. The impact of exposure to pH below physiological range was analyzed by incubation of the cells in MES buffered saline at pH 5.0 for 30 min, which resulted in a reduction in viability to ˜64%, 8% and 33% for L929 fibroblasts, hNPCs and hMSCs, respectively. Microencapsulation in Alg/Alg-DSP/SF-TA microbeads provided protection against 30 min incubation at pH 5.0 as the viabilities of L929 fibroblasts, hNPCs and hMSCs were ˜79%, 70% and 82% after treatment, respectively.

UV shielding of the composite microbeads was assessed by exposing cell-laden beads to UV-C irradiation at 254 nm for 10-, 20-, or 30-min. Quantification of cell viability 2 h after UV irradiation showed that cytotoxicity of the treatment increased with duration. Survival rates decreased from ˜74% to 58% and 48% for L929 fibroblasts; from ˜64% to 61% and 50% for hNPCs; and from ˜80% to 50% and 39% for hMSCs when exposure time was increased from 10 min to 20 min and 30 min, respectively. For L929 fibroblasts and hNPCs encapsulated in dual crosslinked Alg/Alg-DSP/SF-TA hydrogel microbeads, however, viability after UV irradiation was around 75% and 80%, respectively, and no significant difference was recorded among various exposure times. Although the viability of hMSCs gradually decreased from 78% to 70% when the exposure time was increased from 10 to 30 min, cell survival was significantly higher compared to the controls.

Live/dead imaging of hNPCs 48 h after UV treatment for 20 min revealed that almost all cells in the control groups died, suggesting apoptosis in later time points, while many cells in the microencapsulated group were viable and able to spread, confirming the UV shielding effect of the composite microbeads.

Immunocamouflage of microencapsulated hMSCs was assessed by antibody binding to surface markers. hMSCs population defined by side versus forward scatter plot was negative for FITC signal from secondary antibody for secondary antibody only or isotype controls, while it was positive after labeling with anti-CD44 or anti-CD90 antibodies, indicating specific binding to and positivity for CD44 and CD90 surface markers. Confocal micrographs showed that the surfaces of individual hMSCs labeled with anti-CD44 and anti-CD90 antibodies were positive for the FITC signal from the secondary antibody, further confirming positivity of the cells for the surface markers. Microencapsulated cells, on the other hand, were negative for fluorescent signal after incubation with primary and FITC-conjugated secondary antibody, suggesting lack of antibody binding to cell surfaces.

1. A composition comprising:
a composite microbead having embedded therein one or more animal cells, the composite microbead having a material matrix that is composed of silk fibroin and a mixture of modified and unmodified alginate, wherein the composite microbead has reduced disintegration by ion exchange when compared with a comparison microbead,
wherein the comparison microbead replaces the modified alginate with an equal amount by weight of the unmodified alginate, such that the comparison microbead includes no unmodified alginate,
wherein the silk fibroin, the modified alginate, and the unmodified alginate are covalently and ionically crosslinked,
wherein the one or more animal cells can be on-demand released from the composite microbead by contacting the composite microbead with a mixture of a calcium chelator and a reducing agent.

2. The composition of claim 1, wherein the composite microbead has a diameter of between 100 μm and 500 μm.

3. The composition of claim 1, wherein the composite microbead has an elastic modulus that is greater than an elastic modulus of the comparison microbead, wherein applying a compressive force to the composite microbead that would otherwise be fatal to the one or more animal cells in isolation from the composite microbead is not fatal to the one or more animal cells within the composite microbead.

4. The composition of claim 1, wherein the composite microbead provides permselectivity against higher molecular weight molecules.

5. The composition of claim 1, wherein the modified alginate is an alginate conjugated with disulfide-linked phenol moieties.

6. The composition of claim 1, wherein the silk fibroin is a modified silk fibroin including tyrosine and/or tyramine substitution.

7. The composition of claim 1, wherein the material matrix comprises: the unmodified alginate in an amount by weight excluding water of the material matrix of between 10% and 40%, the modified alginate in an amount by weight excluding water of the material matrix of between 10% and 50%, and the silk fibroin in an amount by weight excluding water of the material matrix of between 30% and 75%.

8. The composition of claim 1, the material matrix further comprising a tyramine-modified gelatin, wherein the silk fibroin, the modified alginate, the unmodified alginate, and the tyramine-modified gelatin are covalently crosslinked.

9. The composition of claim 8, wherein the material matrix comprises: the alginate in an amount by weight excluding water of the material matrix of between 10% and 40%, the modified alginate in an amount by weight excluding water of the material matrix of between 10% and 50%, the silk fibroin in an amount by weight excluding water of the material matrix of between 30% and 75%, and the tyramine-modified gelatin in an amount by weight excluding water of the material matrix of between 1% and 40%.

10. The composition of claim 1, wherein the one or more animal cells are L929 murine fibroblasts.

11. The composition of claim 1, wherein the one or more animal cells are bone marrow-derived human mesenchymal stem cells.

12. The composition of claim 1, wherein the one or more animal cells are neural progenitor cells.

13. The composition of claim 1, wherein the composite microbead at least partly prevents binding to surface markers on the one or more animal cells.

14. A method of microencapsulating animal cells, the method comprising:
a) suspending a plurality of the animal cells in a pre-hydrogel solution comprising silk fibroin, modified alginate, and unmodified alginate and optionally further comprising a tyramine-modified gelatin;
b) introducing microdroplets of the pre-hydrogel solution into a crosslinking solution, thereby microencapsulating at least a portion of the plurality of the animal cells into a composite microbead,
wherein the pre-hydrogel solution and/or the crosslinking solution includes horseradish peroxidase,
wherein the crosslinking solution comprises an ionic crosslinker.

15. The method of claim 14, the method further comprising subjecting the composite microbead to elevated pressure.

16. The method of claim 15, wherein subjecting the composite microbead to elevated pressure comprises processing a recombinant protein, transplanting the composite microbead, tissue engineering with the composite microbead, injection-based delivery of the composite microbead, bioprinting the composite microbead.

17. The method of claim 14, the method further comprising culturing the at least a portion of the plurality of the animal cells microencapsulated within the composite microbead.

18. The method of claim 17, wherein the culturing is performed in the presence of polycationic polyethyleneimine, wherein the culturing is performed in the presence of apoptotic inflammatory cytokine tumor necrosis factor (TNF)-α, and/or wherein the culturing is performed at extracellular acidosis at a pH of 5.0.

19. The method of claim 14, wherein the introducing of step b) includes forming the microdroplets with centrifugal force.

20. The method of claim 14, the method further comprising contacting the composite microbead with sodium citrate and a reducing agent, thereby initiating dissolution of the composite microbead and releasing the at least a portion of the plurality of the animal cells.
